(19) United States
(12) Patent Application Publication
(10) Pub. No.: US 2026/0360517 A1
(43) Pub. Date: Nov. 12, 2026

(54) ARTIFICIAL INTELLIGENCE MODEL MANAGEMENT SYSTEM BASED ON METADATA

(71) Applicant: China National Institute of Standardization, Beijing (CN)

(72) Inventors: Guangzhi Sun, Beijing (CN); Yuan Sui, Beijing (CN); Shumin Wang, Beijing (CN); Shuang Wang, Beijing (CN); Yue Cheng, Beijing (CN)

(21) Appl. No.: 19/727,313

(22) Filed: Jul. 1, 2026

Related U.S. Application Data
(63) Continuation of application No. PCT/CN2025/131523, filed on Oct. 31, 2025.

(30) Foreign Application Priority Data
May 8, 2025 (CN) 202510585066.X

(51) Int. Cl. G16H 30/40; G16H 50/20

(52) U.S. Cl. CPC G16H 30/40; G16H 50/20

(57) ABSTRACT

An artificial intelligence (AI) model management system based on metadata includes: a metadata processing module, configured to collect metadata including continuous metadata and discrete metadata, calculate field center points of the continuous metadata and encode the field center points into a label distribution vector, count a category total number of the discrete metadata, perform encoding to construct a mapping matrix, perform vector concatenation, and perform linear mapping using a linear transformation matrix to convert a concatenated vector into a unified-length latent vector. By Gaussian kernel label distribution encoding of continuous metadata and encoding of discrete metadata, the system can cover a complete range of variables through detailed segmentation, ensure that different features can be fused in a same latent space through a unified metadata latent vector while guaranteeing comparability, and introduce a gradual denoising diffusion process into a model, causing medical data generated in each inference to possess higher diversity.

### CROSS-REFERENCE TO RELATED APPLICATIONS

This application is a continuation of PCT/CN2025/131523, filed on Oct. 31, 2025 and claims priority of Chinese Patent Application No. 202510585066.X, filed on May 8, 2025, the entire contents of which are incorporated herein by reference.

### TECHNICAL FIELD

The present disclosure relates to the technical field of model management, and specifically to an artificial intelligence (AI) model management system based on metadata.

### BACKGROUND

Metadata, serving as “data” describing data, contains information such as data sources, data types, data relationships, and data processing methods. With the continuous advancement of big data technology, enterprises and scientific research institutions increasingly rely on efficient metadata management systems when processing massive information. In the medical field, prediction and analysis based on metadata have also been gradually proposed and applied. These systems can not only improve data query efficiency but also reveal deep-seated value hidden behind data through correlation analysis of metadata.

However, existing metadata management methods and artificial intelligence (AI) model training methods still possess certain deficiencies. Current mainstream technologies mostly focus on processing and modeling single-type metadata. The differential processing for continuous metadata and discrete metadata often fails to achieve sufficient refinement. Particularly for medical information, potential relationships among categories are difficult to capture efficiently, resulting in information loss and limited model expression capability. In addition, existing methods usually neglect how to efficiently fuse heterogeneous data sources. Under multi-modal data circumstances in the medical field, model training has difficulty in simultaneously considering data richness and data integrity. Therefore, existing technologies face considerable performance bottlenecks and application limitations when processing complex data types and large-scale data.

### SUMMARY

In view of the above existing problems, the present disclosure is provided.

Therefore, the present disclosure provides an artificial intelligence (AI) model management system based on metadata to solve the problem that differential processing for continuous metadata and discrete metadata often fails to achieve sufficient refinement, and particularly for medical information, potential relationships among categories are difficult to capture efficiently, resulting in information loss and limited model expression capability. In addition, existing methods usually neglect how to efficiently fuse heterogeneous data sources, particularly under multi-modal data circumstances in the medical field, and model training has difficulty in simultaneously considering data richness and data integrity.

To solve the above technical problems, the present disclosure provides the following technical solutions:

In a first aspect, the present disclosure provides an AI model management system based on metadata, including:
a metadata processing module, configured to collect metadata including continuous metadata and discrete metadata, calculate field center points of the continuous metadata and encode the field center points into a label distribution vector, count a category total number of the discrete metadata, perform encoding to construct a mapping matrix, perform vector concatenation, and perform linear mapping using a linear transformation matrix to convert a concatenated vector into a unified-length latent vector;
a medical data processing module, configured to extract an image latent vector of a medical image, fuse the image latent vector with the unified latent vector to generate a conditional vector, introduce, based on external structural features, a control module (ControlNet) to generate a control vector, fuse the control vector with the image latent vector to generate a fused latent vector, gradually add noise and construct a diffusion model based on a U-Net network, and perform reverse denoising training to generate a restored latent vector;
a model deployment module, configured to deploy, by a client, the diffusion model to perform data standardization, aggregate model parameters, update a global model, and evaluate the global model; and
a query module, configured to perform model uncertainty analysis on the global model, define a knowledge graph (KG), record model operation data, upload an operation log, convert a query statement input by a user, and perform a data query.

In a preferred solution of the AI model management system based on metadata according to the present disclosure: the performing vector concatenation, and performing linear mapping using a linear transformation matrix to convert a concatenated vector into a unified-length latent vector includes,
for each field of type continuous metadata, calculating a center point of each field according to a variable range and score segments of field sample values;
constructing a Gaussian kernel label distribution function to encode a value thereof into a vector, and mapping an input value x of the field into the label distribution vector;
for each field of type discrete metadata, counting the number of unique values of all samples of the field as a category total number C, and performing Embedding encoding to construct the mapping matrix with a dimension of C×d, where d is a fixed embedding dimension;
with each row representing an embedding vector of a category, obtaining an embedding vector representation of each discrete variable;
concatenating the label distribution vectors and the embedding vector representations of the discrete metadata, in which a dimension of output vector of all continuous variables is p, a dimension of embedding vectors of all discrete variables is d, and a concatenated dimension D is defined according to a continuous variable total number N_{c} and a discrete variable total number N_{d}; and
sequentially concatenating encoded vectors of all the fields into a total vector, and performing the linear mapping using the transformation matrix to convert the total vector into the unified-length latent vector.

In a preferred solution of the AI model management system based on metadata according to the present disclosure: the generating a control vector, and fusing the image latent vector with the unified latent vector to generate a conditional vector includes,
based on medical image data corresponding to clinical data, pairing the image data with the unified latent vector of the metadata, extracting an image latent vector of the medical image data using a pretrained image encoder, and fusing the image latent vector with the unified latent vector of the metadata to generate a conditional vector;
determining external structural features of the medical image data, the external structural features including key points, labels, and structural information of an image, introducing a ControlNet, inputting the conditional vector and the external structural features to generate a control vector zₚ, and fusing the control vector with the image latent vector to generate a fused latent vector;
performing a forward diffusion process based on the fused latent vector through gradual noise addition;
constructing a diffusion model based on a U-Net network, performing reverse denoising training to restore a latent vector of an original image from a noise state, and training the model to predict noise using a mean square error (MSE) loss;
performing gradient descent optimization using an Adam optimizer, updating model parameters, and stopping iteration when a loss of the model no longer decreases significantly during successive iterations; and
generating a condition based on a noise input in a latent space and the conditional vector, and generating a restored latent vector through the trained diffusion model.

In a preferred solution of the AI model management system based on metadata according to the present disclosure: the aggregating model parameters and updating a global model includes,
collecting client metadata according to client terminals, standardizing metadata of each client using a metadata calibrator, Meta-Calibrator, deploying the diffusion model, and performing model training using local data; and
determining a weighting coefficient for each client, aggregating the model parameters of all the clients using a weighted averaging method, and updating the global model according to global model parameters.

In a preferred solution of the AI model management system based on metadata according to the present disclosure: the evaluating the global model includes,
performing a Fréchet inception distance (FID) evaluation and a structural similarity index measure (SSIM) evaluation on the global model;
calculating, as a difference threshold, a sum of a mean and a standard deviation of historical distribution difference values between generated images and real images according to the evaluation, and determining that original features of an image are retained when a distribution difference value is less than or equal to the difference threshold; and
calculating, as a structure threshold, a sum of a mean and a standard deviation of historical structure difference values between generated images and real images according to the evaluation, and determining that original features of an image are retained when a structure difference value is greater than or equal to the structure threshold.

In a preferred solution of the AI model management system based on metadata according to the present disclosure: the performing model uncertainty analysis on the global model includes,
introducing a repeated sampling mechanism (LAS) for the global model, performing multiple random initializations on an input image latent vector and the conditional vector, obtaining corresponding repeated sampling restored latent vectors, and calculating an average predicted value and an average standard deviation to generate an uncertainty score; and
calculating, as a score threshold, a sum of a mean and twice a standard deviation of historical uncertainty scores, and determining that uncertainty is low when the uncertainty score is less than the score threshold.

In a preferred solution of the AI model management system based on metadata according to the present disclosure: the collecting metadata including continuous metadata and discrete metadata includes,
traversing each column field in an original clinical data table, reading a value type and a value distribution range of each column, marking the field as continuous metadata when a field value is of a continuous numeric type, marking the field as discrete metadata when a field value is a discrete classification label, and using each row in the clinical data table as a sample.

In a preferred solution of the AI model management system based on metadata according to the present disclosure: the defining a KG and recording model operation data refers to including: defining a triple structure of the KG using a DataHub architecture, and recording input/output mappings, data sources, processing steps, model parameters, and execution environment information of the model.

In a preferred solution of the AI model management system based on metadata according to the present disclosure: the uploading an operation log refers to including: recording an operation log for each operation step of the model, and storing the operation log as structured metadata.

In a preferred solution of the AI model management system based on metadata according to the present disclosure: the converting a query statement input by a user and performing a data query includes,
converting the query statement input by the user into a SPARQL statement using a BERT large model, and querying the metadata in the KG; and
according to the user query, returning, by the system, a corresponding model path, an inference condition, and detailed information of input data.

Beneficial effects of the present disclosure are: by Gaussian kernel label distribution encoding of continuous metadata and encoding of discrete metadata, a complete range of variables can be covered through detailed segmentation; a unified metadata latent vector ensures that features of different types can be fused in a same latent space while guaranteeing comparability of the features; and a gradual denoising diffusion process is introduced into the model, causing medical data generated in each inference to possess higher diversity. The introduction of noise allows the model to generate diverse latent vector representations, enhancing flexibility of the model in actual clinical applications, particularly in disease progression prediction, personalized medicine, and simulation of different clinical trials, thereby providing more prediction options.

### BRIEF DESCRIPTION OF THE DRAWINGS

To more clearly illustrate technical solutions of embodiments of the present disclosure, drawings required in the description of the embodiments are briefly introduced below. Evidently, drawings in the following description are merely some embodiments of the present disclosure. For a person of ordinary skill in the art, other drawings may be obtained according to these drawings without creative efforts.

FIG. 1 is a schematic structural diagram of an artificial intelligence (AI) model management system based on metadata according to Embodiment 1; and

FIG. 2 is a schematic flow diagram of the AI model management system based on metadata according to Embodiment 1.

### DETAILED DESCRIPTION

To make the above objects, features, and advantages of the present disclosure more obvious and comprehensible, specific implementations of the present disclosure are described in detail below with reference to the accompanying drawings of the specification.

Many specific details are set forth in the following description to facilitate a full understanding of the present disclosure. However, the present disclosure may also be implemented in other ways different from the ways described herein. A person skilled in the art may make similar generalizations without departing from the spirit of the present disclosure. Therefore, the present disclosure is not limited to specific embodiments disclosed below.

Furthermore, “one embodiment” or “embodiment” referred to herein means a specific feature, structure, or characteristic that may be contained in at least one implementation of the present disclosure. “In one embodiment” appearing in different places in the present specification does not refer to the same embodiment, nor is a separate or alternative embodiment mutually exclusive with other embodiments.

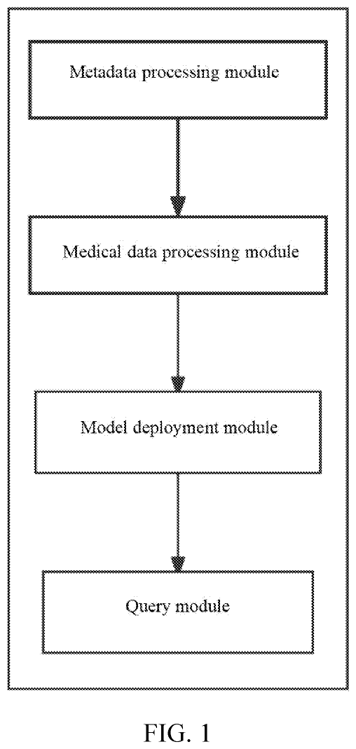

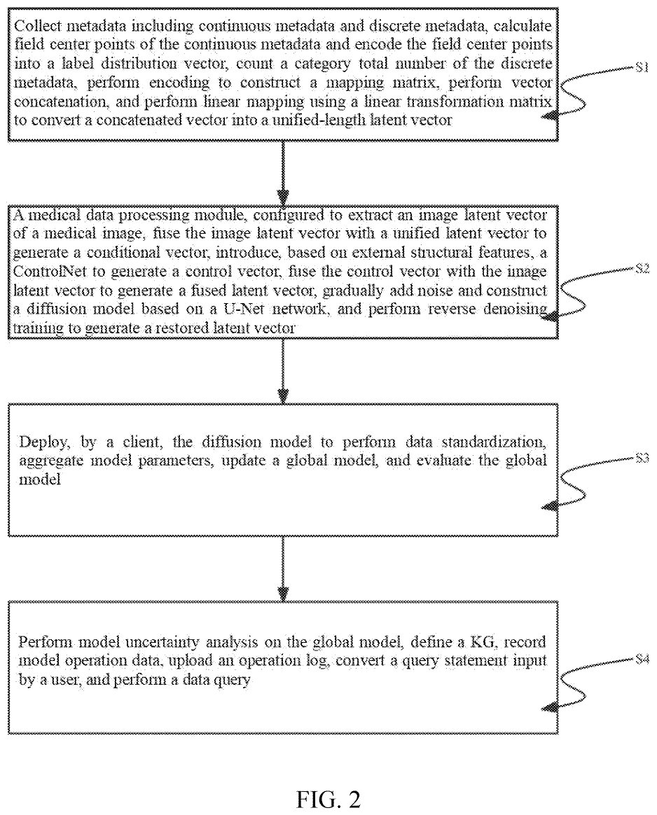

Embodiment 1, referring to FIG. 1 to FIG. 2, is a first embodiment of the present disclosure. This embodiment provides an artificial intelligence (AI) model management system based on metadata, including the following steps:

In S1, metadata including continuous metadata and discrete metadata is collected, field center points of the continuous metadata are calculated and encoded into a label distribution vector, a category total number of the discrete metadata is counted, encoding is performed to construct a mapping matrix, vector concatenation is performed, and linear mapping is performed using a linear transformation matrix to convert a concatenated vector into a unified-length latent vector.

Preferably, metadata including continuous metadata and discrete metadata being collected includes,
each column field in an original clinical data table is traversed, a value type and a value distribution range of each column are read, the field is marked as continuous metadata when a field value is of a continuous numeric type, the field is marked as discrete metadata when a field value is a discrete classification label, and each row in the clinical data table is used as a sample.

By traversing the original clinical data table and performing classification according to the value type and the value distribution range of each column, data can be effectively and automatically marked as continuous metadata or discrete metadata, facilitating clear identification of a data structure and providing accurate data type identifiers for subsequent data processing, modeling, and analysis, thereby ensuring consistency and efficiency during processing while helping a model adopt appropriate processing methods according to characteristics of data when processing data of different types, thus improving performance and accuracy of the model.

Further, vector concatenation being performed, and linear mapping being performed using a linear transformation matrix to convert a concatenated vector into a unified-length latent vector includes,
for each field of type continuous metadata, a center point of each field is calculated according to a variable range and score segments of field sample values, expressed as:

$${μ}_{i} = \frac{i}{n - 1} \cdot ( b - a ) + a ,$$

where μᵢ denotes an ith Gaussian kernel center point, n denotes the number of segments, b denotes a maximum value of the variable range, and a denotes a minimum value of the variable range;
a Gaussian kernel label distribution function is constructed to encode a value thereof into a vector, and an input value x of the field is mapped into the label distribution vector, expressed as:

$${v}_{i} = exp ( - \frac{{( x - {μ}_{i} )}^{2}}{2 {σ}^{2}} ) ,$$

where vᵢ denotes a label distribution vector of an ith field, and σ denotes a smoothing factor, which is determined according to a formula

$$σ = \frac{b - a}{2 n}$$

to ensure consistent distribution coverage width;
for each field of type discrete metadata, the number of unique values of all samples of the field is counted as a category total number C, and Embedding encoding is performed to construct the mapping matrix with a dimension of C×d, where d is a fixed embedding dimension;
with each row representing an embedding vector of a category, an embedding vector representation of each discrete variable is obtained; and for each sample, a category index j is obtained through table lookup, and a jth row is extracted as an embedding vector;

the label distribution vectors and the embedding vector representations of the discrete metadata are concatenated, in which a dimension of output vectors of all continuous variables is p, a dimension of embedding vectors of all discrete variables is d, and a concatenated dimension D is defined according to a continuous variable total number N_{c} and a discrete variable total number N_{d}, expressed as: D=N_{c}·p+N_{d}·d; and
encoded vectors of all the fields are sequentially concatenated into a total vector, and the linear mapping is performed using the linear transformation matrix to convert the total vector into the unified-length latent vector, in which the vector is a metadata conditional representation and is fed into an AI model control path, expressed as:

$${v}_{me}^{r} = {v}_{1} ‖ {v}_{2} ‖ ⋯ ‖ {v}_{{N}_{c} + {N}_{d}} ,$$

$$and$$

$${v}_{m} = {v}_{me}^{r} \cdot W ,$$

where

$${v}_{m e}^{r}$$

denotes a raw vector after concatenation of all metadata, a length being D, v_{N}_{_{c}}_{+N}_{_{d}} denotes an encoded vector of an N_{c}+N_{d}th field, vₘ denotes a unified latent vector representation of the metadata, and W denotes a linear transformation matrix, which is initialized through standard normal distribution and is fixed during training.

By Gaussian kernel label distribution encoding of continuous metadata and Embedding encoding of discrete metadata, a complete range of variables can be covered through detailed segmentation, avoiding information loss caused by a single numerical input and allowing the model to more accurately learn underlying patterns of data when processing features obtained from the encoding. For discrete metadata, Embedding encoding, by mapping each category into a dense vector space, can effectively reduce dimensionality while retaining category information, thereby improving generalization ability and prediction accuracy of the model.

A unified metadata latent vector ensures that features of different types can be fused in a same latent space, while guaranteeing comparability of the features, and relative relationships among the features can be captured by the model, thereby providing a more comprehensive conditional input for the model. The unified metadata latent vector representation not only improves the degree of data integration but also enhances a comprehensive understanding capability of the model for various input features, thus further improving performance of generation tasks or prediction tasks.

In S2, an image latent vector of a medical image is extracted, and the image latent vector is fused with the unified latent vector to generate a conditional vector, a control module (ControlNet) is introduced based on external structural feature to generate a control vector, the control vector is fused with the image latent vector to generate a fused latent vector, noise is gradually added and a diffusion model is constructed based on a U-Net network, and reverse denoising training is performed to generate a restored latent vector.

Preferably, a control vector being generated, and the image latent vector being fused with the unified latent vector to generate a conditional vector includes,
based on medical image data corresponding to clinical data, the image data is paired with the unified latent vector of the metadata, an image latent vector of the medical image data is extracted using a pretrained image encoder and is fused with the unified latent vector of the metadata to generate a conditional vector, expressed as:

z_{c}=γ·zᵢ+(1+γ)·vₘ,
where z_{c} denotes a conditional vector, zᵢ denotes an image latent vector output by an image encoder, and γ denotes a fusion coefficient, which is determined based on historical experience;
external structural features of the medical image data are determined, the external structural features including key points, labels, and structural information of an image, a ControlNet is introduced, the conditional vector and the external structural feature are input to generate a control vector zₚ, and the control vector is fused with the image latent vector to generate a fused latent vector, expressed as:

z_{co}=β·zᵢ+(1−β)·zₚ,
where z_{co} denotes a fused latent vector, and β denotes a control weight, which is obtained through training optimization based on calibrated training data;
based on the fused latent vector, a forward diffusion process is performed through gradual noise addition, so that each image latent vector becomes more blurred, expressed as:

$${z}_{co , t} = \sqrt{{a}_{t}} {z}_{co , t - 1} + \sqrt{1 - {a}_{t}^{2}} ϵ ,$$

where z_{co,t} and z_{co,t-1} denote fused latent vectors at time step t and time step t−1, aₜ denotes an attenuation factor at a tth step, which is determined based on historical experience, and ∈ denotes standard Gaussian noise, noise having a covariance that is an identity matrix;
a diffusion model is constructed based on a U-Net network, reverse denoising training is performed to restore a latent vector of an original image from a noise state, and the model is trained to predict noise using a mean square error (MSE) loss, expressed as:

$${L}_{n} = {‖ {ϵ}_{θ} ( {z}_{t} , t , {z}_{c} ) - ϵ ‖}_{2}^{2} ,$$

where Lₙ denotes a calculated loss, ∈_{θ} denotes a U-Net network model for predicting noise;
gradient descent optimization is performed using an Adam optimizer, model parameters are updated, iteration is stopped when a loss of the model no longer decreases significantly during successive iterations; and
a condition is generated based on a noise input in a latent space and the conditional vector, and a restored latent vector is generated through the trained diffusion model and is reconstructed as image data through a decoder.

By pairing medical data with the unified-length latent vector of the metadata, the generated conditional vector can provide more comprehensive input information for the model, allowing the model to simultaneously consider latent features of the medical data and personalized metadata of a patient. By controlling the conditional vector through the ControlNet module, a fine-grained control capability of the model over the medical data can be further enhanced. Particularly in medical data generation tasks, controlling the influence of external structural features can help the model generate medical data that better conforms to medical knowledge, for example, accurately reconstructing anatomical structures of a specific part, or adjusting certain features of the medical data (such as lesion regions). Through optimized control weights, the model can more precisely adjust the generation process based on feedback from training data, ensuring that the generated images better meet clinical needs.

The forward diffusion process is performed on the fused latent vector, and noise is added, allowing the model to learn how to gradually recover from a blurred state, thereby being able to better generate high-quality medical data when facing unknown or blurred inputs. The introduction of the diffusion model can further improve the diversity of generated medical data, and allow the model to generate different versions of medical data, enhancing the exploration capability of the model.

The diffusion model constructed based on the U-Net network, through reverse denoising training, can allow the model to gradually restore a clear image latent vector from noise, and the model can accurately predict image details during denoising, while maintaining an efficient training process. The Adam optimizer is used, which ensures that the model parameters can be updated stably, thereby accelerating convergence and improving the performance of the model.

The trained diffusion model can generate the restored latent vector based on the noise input and the conditional vector, and transform the restored latent vector into image data through the decoder. By merging the medical image data and the metadata to generate the conditional vector, personalized information of a patient can be effectively introduced into the image generation process. The introduction of the ControlNet can cause the external structural features to be considered in the image generation process, and can precisely control a specific anatomical structure during the generation process, effectively reducing the distortion of generated images and improving the medical credibility of the images.

By introducing the gradual denoising diffusion process into the model, the medical data generated in each inference possesses higher diversity. The introduction of noise allows the model to generate diverse latent vector representations, thereby outputting different versions of the medical data, enhancing flexibility of the model in actual clinical applications, particularly in disease progression prediction, personalized medicine, and simulation of different clinical trials, thereby providing more prediction options.

In S3, the diffusion model is deployed by a client to perform data standardization, model parameters are aggregated, a global model is updated, and the global model is evaluated.

Preferably, model parameters being aggregated and a global model being updated includes,
client metadata is collected according to client terminals, metadata of each client is standardized using a metadata calibrator, Meta-Calibrator, the diffusion model is deployed, and model training is performed using local data; and
a weighting coefficient is determined for each client, the model parameters of all the clients are aggregated using a weighted averaging method, the global model is updated according to global model parameters, expressed as:

$${θ}_{g} = {\sum }_{i}^{M} \frac{\vert {D}_{i} \vert}{\vert D \vert} \cdot {θ}_{i} ,$$

where θg denotes global model parameters, M denotes a total client number, Dᵢ denotes a data volume of a client i, D denotes a total data volume, θᵢ denotes parameters of an ith client model, including weights and biases of the diffusion model, and

$$\frac{\vert {D}_{i} \vert}{\vert D \vert}$$

serves as model weights of different clients.

The use of the Meta-Calibrator can ensure that all client data is processed under the same standard, thereby improving data uniformity and consistency. Deploying the diffusion model and performing training using local data can allow each client to perform a personalized adjustment according to its own local data, thereby improving model performance of each client. The model parameters of the clients are aggregated using the weighted averaging method, helping to better balance the influence of each client.

Further, the global model being evaluated includes,
a Fréchet inception distance (FID) evaluation and a structural similarity index measure (SSIM) evaluation are performed on the global model;
a sum of a mean and a standard deviation of historical distribution difference values between generated images and real images according to the evaluation is calculated as a difference threshold, and it is determined that original features of an image are retained when a distribution difference value is less than or equal to the difference threshold; and
a sum of a mean and a standard deviation of historical structure difference values between generated images and real images according to the evaluation is calculated as a structure threshold, and it is determined that original features of an image are retained when a structure difference value is greater than or equal to the structure threshold.

By evaluating the differences between the generated images and the real images using FID and SSIM, the improvement of image quality and fidelity can be effectively measured. The FID evaluation can quantify the distribution difference between the generated images and the real images, while the SSIM evaluation focuses on the structural similarity of the images.

In S4, model uncertainty analysis is performed on the global model, a knowledge graph (KG) is defined, model operation data is recorded, an operation log is uploaded, a query statement input by a user is converted, and a data query is performed.

Preferably, model uncertainty analysis being performed on the global model includes,
a repeated sampling mechanism (LAS) is introduced for the global model, multiple random initializations are performed on an input image latent vector and the conditional vector, and outputs of the model under different initialization conditions are obtained through multiple sampling, thereby obtaining uncertainty information of results; and corresponding repeated sampling restored latent vectors are obtained, and an average predicted value and an average standard deviation are calculated to generate an uncertainty score; and
a sum of a mean and twice a standard deviation of historical uncertainty scores is calculated as a score threshold, and it is determined that uncertainty is low when the uncertainty score is less than the score threshold.

By introducing the LAS, the model can perform multiple random initializations on the input image latent vector and the conditional vector, obtain the outputs under different initialization conditions, and thereby better reflect the uncertainty of generated results.

By calculating the average predicted value and the average standard deviation of restored latent vectors obtained through multiple sampling, an uncertainty score can be generated for each output, thereby effectively identifying which generated results have higher stability and which have greater uncertainty.

Further, a KG being defined and model operation data being recorded includes,
a triple structure of the KG is defined using a DataHub architecture, and input/output mappings, data sources, processing steps, model parameters, and execution environment information of the model are recorded. Each triple contains: entities (such as datasets, models, and inference results), attributes (such as data types, calculation modes, and model states), and relationships (such as mapping relationships between models and datasets).

By defining the triple structure of the KG using the DataHub architecture, the input/output mappings, data sources, processing steps, model parameters, and execution environment information of the model can be systematically recorded and managed. This structured information storage manner provides a clear framework for the traceability, management, and analysis of data, ensuring the transparency of data flow and model operation. The form of triples makes the definitions of entities, attributes, and relationships more explicit, facilitating collaboration and information sharing across systems or teams, thereby improving the traceability, maintainability, and efficiency of the workflow.

Further, an operation log being uploaded refers to including that: an operation log is recorded for each operation step of the model, and is stored as structured metadata for audit and review.

All the metadata is indexed and associated with a specific training process, an inference result, or an abnormal event. Using a graph query interface, all operation history related to a certain model or dataset can be quickly looked up by a user.

By defining the triple structure of the KG using the DataHub architecture, the input/output mappings, data sources, processing steps, model parameters, and execution environment information of the model can be systematically recorded and managed. This structured information storage manner provides a clear framework for the traceability, management, and analysis of data, ensuring the transparency of data flow and model operation. The form of triples makes the definitions of entities, attributes, and relationships more explicit, facilitating collaboration and information sharing across systems or teams, thereby improving the traceability, maintainability, and efficiency of the workflow.

Further, a query statement input by a user being converted and a data query being performed includes,
the query statement input by the user is converted into a SPARQL statement using a BERT large model, and the metadata in the KG is queried; and
according to the user query, a corresponding model path, an inference condition, and detailed information of input data are returned by the system.

By converting the query statement input by the user into the SPARQL statement using the BERT large model, more natural and intelligent user interaction can be achieved, causing the user to obtain information related to the metadata in the KG through a simple query. This automated conversion improves the flexibility and efficiency of the query, allowing the system to quickly return, according to user needs, related model paths, inference conditions, and detailed information of the input data, thereby optimizing the application of the KG and the user experience.

In summary, the present disclosure, by Gaussian kernel label distribution encoding of continuous metadata and encoding of discrete metadata, can cover a complete range of variables through detailed segmentation, ensure that features of different types can be fused in a same latent space through a unified metadata latent vector while guaranteeing comparability of the features, and introduce a gradual denoising diffusion process into the model, causing medical data generated in each inference to possess higher diversity. The introduction of noise allows the model to generate diverse latent vector representations, enhancing flexibility of the model in actual clinical applications, particularly in disease progression prediction, personalized medicine, and simulation of different clinical trials, thereby providing more prediction options.

It is to be noted that the above embodiments are merely for illustrating the technical solutions of the present disclosure rather than as a limitation. Although the present disclosure has been described in detail with reference to preferred embodiments, those of ordinary skill in the art understand that modifications or equivalent replacements may be made to the technical solutions of the present disclosure without departing from the spirit and scope of the technical solutions of the present disclosure, and such modifications and equivalent replacements all fall within the scope of the claims of the present disclosure.

1. An artificial intelligence (AI) model management system based on metadata, comprising:
a metadata processing module, configured to collect metadata comprising continuous metadata and discrete metadata, calculate field center points of the continuous metadata and encode the field center points into a label distribution vector, count a category total number of the discrete metadata, perform encoding to construct a mapping matrix, perform vector concatenation, and perform linear mapping using a linear transformation matrix to convert a concatenated vector into a unified-length latent vector;
a medical data processing module, configured to, based on medical image data corresponding to clinical data, pair the image data with the unified latent vector of the metadata, extract an image latent vector of the medical image data using a pretrained image encoder, and fuse the image latent vector with the unified latent vector of the metadata to generate a conditional vector;
determine external structural features of the medical image data, the external structural features comprising key points, labels, and structural information of an image, introduce a control module (ControlNet), input the conditional vector and the external structural features to generate a control vector zₚ, and fuse the control vector with the image latent vector to generate a fused latent vector;
perform a forward diffusion process based on the fused latent vector through gradual noise addition;
construct a diffusion model based on a U-Net network, perform reverse denoising training to restore a latent vector of an original image from a noise state, and train the model to predict noise using a mean square error (MSE) loss;
perform gradient descent optimization using an Adam optimizer, update model parameters, and stop iteration when a loss of the model no longer decreases significantly during successive iterations; and
generate a condition based on a noise input in a latent space and the conditional vector, and generate a restored latent vector through the trained diffusion model;
a model deployment module, configured to deploy, by a client, the diffusion model to perform data standardization, aggregate the model parameters, update a global model, and evaluate the global model; and
a query module, configured to perform model uncertainty analysis on the global model, define a knowledge graph (KG), record model operation data, upload an operation log, convert a query statement input by a user, and perform a data query.

2. The AI model management system based on metadata according to claim 1, wherein the performing vector concatenation, and performing linear mapping using a linear transformation matrix to convert a concatenated vector into a unified-length latent vector comprises, for each field of type continuous metadata, calculating a center point of each field according to a variable range and score segments of field sample values;
constructing a Gaussian kernel label distribution function to encode a value thereof into a vector, and mapping an input value x of the field into the label distribution vector;
for each field of type discrete metadata, counting the number of unique values of all samples of the field as a category total number C, and performing Embedding encoding to construct the mapping matrix with a dimension of C×d, where d is a fixed embedding dimension;
with each row representing an embedding vector of a category, obtaining an embedding vector representation of each discrete variable;
concatenating the label distribution vectors and the embedding vector representations of the discrete metadata, wherein a dimension of output vectors of all continuous variables is p, a dimension of embedding vectors of all discrete variables is d, and a concatenated dimension D is defined according to a continuous variable total number N_{c} and a discrete variable total number N_{d}; and
sequentially concatenating encoded vectors of all the fields into a total vector, and performing the linear mapping using the linear transformation matrix to convert the total vector into the unified-length latent vector.

3. The AI model management system based on metadata according to claim 2, wherein the aggregating the model parameters and updating a global model comprises,
collecting client metadata according to client terminals, standardizing metadata of each client using a metadata calibrator, Meta-Calibrator, deploying the diffusion model, and performing model training using local data; and
determining a weighting coefficient for each client, aggregating the model parameters of all the clients using a weighted averaging method, and updating the global model according to global model parameters.

4. The AI model management system based on metadata according to claim 3, wherein the evaluating the global model comprises,
performing a Fréchet inception distance (FID) evaluation and a structural similarity index measure (SSIM) evaluation on the global model;
calculating, as a difference threshold, a sum of a mean and a standard deviation of historical distribution difference values between generated images and real images according to the evaluation, and determining that original features of an image are retained when a distribution difference value is less than or equal to the difference threshold; and
calculating, as a structure threshold, a sum of a mean and a standard deviation of historical structure difference values between generated images and real images according to the evaluation, and determining that original features of an image are retained when a structure difference value is greater than or equal to the structure threshold.

5. The AI model management system based on metadata according to claim 4, wherein the performing model uncertainty analysis on the global model comprises,
introducing a repeated sampling mechanism (LAS) for the global model, performing multiple random initializations on an input image latent vector and the conditional vector, obtaining corresponding repeated sampling restored latent vectors, and calculating an average predicted value and an average standard deviation to generate an uncertainty score; and
calculating, as a score threshold, a sum of a mean and twice a standard deviation of historical uncertainty scores, and determining that uncertainty is low when the uncertainty score is less than the score threshold.

6. The AI model management system based on metadata according to claim 5, wherein the collecting metadata comprising continuous metadata and discrete metadata comprises,
traversing each column field in an original clinical data table, reading a value type and a value distribution range of each column, marking the field as continuous metadata when a field value is of a continuous numeric type, marking the field as discrete metadata when a field value is a discrete classification label, and using each row in the clinical data table as a sample.

7. The AI model management system based on metadata according to claim 6, wherein the defining a KG and recording model operation data refers to comprising: defining a triple structure of the KG using a DataHub architecture, and recording input/output mappings, data sources, processing steps, model parameters, and execution environment information of the model.

8. The AI model management system based on metadata according to claim 7, wherein the uploading an operation log refers to comprising: recording an operation log for each operation step of the model, and storing the operation log as structured metadata.

9. The AI model management system based on metadata according to claim 8, wherein the converting a query statement input by a user and performing a data query comprises,
converting the query statement input by the user into a SPARQL statement using a BERT large model, and querying the metadata in the KG; and
according to the user query, returning, by the system, a corresponding model path, an inference condition, and detailed information of input data.
